(19) United States
(12) Patent Application Publication
(10) Pub. No.: US 2021/0299308 A1
(43) Pub. Date: Sep. 30, 2021

(54) Disinfection System

(71) Applicant: Airandé PTY Limited, Gordon, New South Wales (AU)

(72) Inventors: William James Hovey, St. Ives (AU); Christopher David Ryman, Lilyfield (AU); Thomas William Jack Hovey, Bathurst (AU); Fabienne Marie-Armande Mertens, Grez Doiceau (BE); Jean-Paul Marie Cornelis, Bruxelles (BE)

(73) Assignee: Airandé PTY Limited, Gordon, New South Wales (AU)

(21) Appl. No.: 17/262,023

(22) PCT Filed: Jul. 24, 2019
(86) PCT No.: PCT/AU2019/050775

§ 371 Date: Jan. 21, 2021

(30) Foreign Application Priority Data
Jul. 24, 2018 (AU) 2018902675

(51) Int. Cl. A61L 2/22; A61L 2/24; B05B 7/24; B05B 12/08; G06K 7/10; G06K 19/07

(52) U.S. Cl. CPC A61L 2/22; A61L 2/24; B05B 7/2491; A61L 2101/02; G06K 7/10366; G06K 19/0723; B05B 12/087

(57) ABSTRACT

The present invention relates to a disinfection system comprising a plurality of replaceable containers, to contain a solution comprising a disinfecting agent, conduits leading from the containers to a manifold, misting apparatus to generate a mist containing the disinfecting agent and to release the mist via exit conduits, through at least one exit vent, wherein the manifold comprises a plenum which receives a volume of air for controlling balance of air pressure as the air is released through the at least one exit vent. The present invention also relates to a method of controlling use of a disinfection system.

### FIELD OF THE INVENTION

The present invention relates to a disinfection system. In particular, embodiments of the invention relate to disinfection systems for decontaminating a volume using a mist generated by the disinfection system.

### BACKGROUND OF THE INVENTION

Contamination in hospitals, clinics, dental surgeries and laboratories poses a significant risk to healthcare workers, lab workers and patients. There are many sources of infection in each of these environments that can result in significant public health and economic costs. For example, the estimated economic cost of healthcare associated infections in the US is US$3.5 billion, and in 2011 there were an estimated 722,000 healthcare associated infections in US acute care hospitals, with roughly 10% of patients with healthcare associated infections dying during their hospitalisation.

Nosocomial (healthcare-acquired) diseases include a plethora of those that are associated with antimicrobial resistant (AMR) microorganisms, including VRE, MRSA, and Pseudomonas aeruginosa, as well as those associated with environmentally persistent opportunistic microorganisms such as Clostridium difficile (a bacterial spore-former) and Candida auris (an emerging bloodstream pathogenic yeast) that are of particular concern for immunocompromised populations.

Besides patients, the risk of transmission of infectious disease among healthcare workers is very real. In healthcare settings the contamination of the personnel can be done either by the patient, by another caregiver or by a contaminated environment, including contaminated instruments, equipment and surfaces. In dental clinics, the dental practitioner operates in a cavity of the human body rich in infectious agents; including Streptococcus pneumonia group A, Staphylococcus aureus, Haemophilus influenza, meningococcus, Herpes simplex, Hepatitis, and Candida albicans. Transmission of infectious agents from patients to staff in the dental setting can occur by direct contact, or by indirect contact with contaminated instruments, equipment or surfaces, as well as through the generation of aerosols (blood, saliva, rinse water) during treatment. In laboratories, the bacteria VRSA, VISA, Salmonella, Shigella, Brucella, C. difficile, Escherichia coli and Klebsiella have all been associated with laboratory-acquired infections. In laboratories transmission of infectious agents can occur through contamination of the air when employing techniques such as vortexing and other work practices that generate aerosols, or may result in inadvertent contamination of surfaces, followed by inadvertent inhalation or ingestion.

Prevention and disinfection are the main elements in the fight against healthcare and laboratory-acquired infections. It is desirable to reduce or remove contamination from the environment and reduce the risk of contamination through bacteria, viruses, spores, yeast and moulds.

Various disinfection agents are used commercially, and one method is to use vapour or mist containing a disinfection agent for non-manual, whole-of-room, broad spectrum decontamination. Examples of disinfection agents include hydrogen peroxide, aldehydes, alcohols, phenolic derivatives, chlorine derivatives and quaternary ammonium cations.

Specifically in relation to hydrogen peroxide vapour, significant research challenges remain to be met. In particular, the effectiveness of hydrogen peroxide vapour for whole-of-room disinfection is highly dependent on penetration, which in turn is highly dependent on misting quality and dispersal. Further, in order to be logistically viable for application in healthcare and laboratory settings, where only minimal downtime is available, optimisation of the rate of misting is a further research engineering challenge, along with misting quality (including particle size).

There exists a need to overcome, or at least alleviate, one or more of the difficulties or deficiencies associated with the prior art.

The present invention aims to address or ameliorate some or all of the above disadvantages in conventional disinfection systems, or at least provide a commercially useful alternative.

### SUMMARY OF THE INVENTION

In an aspect of the present invention, there is provided a disinfection system comprising:
a plurality of replaceable containers, to contain a solution comprising a disinfecting agent;
conduits leading from the containers to at least one exit vent for releasing a mist comprising the disinfection solution;
misting apparatus to generate a mist containing the disinfecting agent and release the mist through the at least one exit vent.

In a preferred embodiment, the present invention provides a disinfection system comprising:
a plurality of replaceable containers, to contain a solution comprising a disinfecting agent;
conduits leading from the containers to a manifold;
misting apparatus to generate a mist containing the disinfecting agent and to release the mist via exit conduits, through at least one exit vent,
wherein the manifold comprises a plenum which receives a volume of air for controlling balance of air pressure as the air is released through the at least one exit vent.

In a preferred embodiment of this aspect of the present invention, the mist may be a dry mist. Preferably, the mist comprises particles having a diameter of between approximately 5 to 15 microns.

The conduits leading from the containers may be liquid conduits, preferably comprising of parts that may be independently rotated relative to one another. The disinfecting agent is preferably hydrogen peroxide. Hydrogen peroxide vapour is more effective than most other existing disinfection modalities, including aldehydes, alcohols, phenolic derivatives, chlorine derivatives and quaternary ammonium cations. Further, hydrogen peroxide vapour offers significant additional advantages over other disinfection modalities, including being environmentally friendly (hydrogen peroxide decomposes into water and oxygen), odourless, leaving little or nil residue, safe (when used according to directions), compatible with most materials found in healthcare and laboratory settings, and efficient and cost effective, saving time and labour in their disinfection routine.

The solution may comprise a low dilution solution. The solution is preferably under 15% hydrogen peroxide. The solution may be: under 14%; under 13%; under 12%; under 11%; under 10%; under 9%; under 8%; under 7%; under 6%; or under 5% hydrogen peroxide. The solution may be in the range of 7% to 8%. In preferred embodiments, dilution solutions of 7.25% and/or 7.9% may be used.

Preferably there are at least two exit vents. The use of two exit vents, and two (or in some embodiments more) containers, provides significant potential for increasing flow rate of disinfectant mist, meaning that the disinfection system of the present invention has a significantly faster operating cycle than existing systems. Each exit vent may comprise a nozzle, a bowl, and apertures in the bowl, to utilise the Venturi effect to draw mist out from the exit conduits, through the nozzle. The mist may be a dry pulverised mist.

Thus, use of the Venturi effect, in relationship with the extended nozzle in the bowl, creates a dry mist in a way that does not rely on a nozzle that might have multiple exit points. By the “Venturi effect” is meant a reduction in fluid pressure that results when a fluid flows through a constricted section (or choke) of a pipe. This in turn involves the creation of a high velocity swirl. The resulting dry mist is capable of reaching substantially all parts of a space to be disinfected, for example a space of a size of at least 50 cubic metres, more preferably at least 100 cubic metres, even more preferably at least 150 cubic metres. The dry mist may be capable of reaching “seen” as well as “unseen” surfaces, for example, the underneath of chairs, operating tables, etc.

The conduits preferably comprise:
a conduit leading from each container to a manifold; wherein the manifold may comprise a plenum, a lower conduit and exit conduits, with the exit conduits leading from the plenum to each exit vent.

The exit conduits may be angled at 30 degrees or more to vertical; 30 to 40 degrees to vertical; or at approximately 35 degrees to vertical.

In another aspect of the present invention, there is provided a disinfection system comprising:
at least one replaceable container, to contain a solution comprising a disinfecting agent, the container having an identifier tag identifying the container;
a conduit leading from the container to at least one exit vent for releasing a mist comprising the disinfection solution;
misting apparatus to generate a mist containing the disinfecting agent and release the mist through the at least one exit vent; and
a scanner to scan the identifier tag, to identify the container.

In a preferred embodiment, there is provided a disinfection system comprising:
at least one replaceable container, to contain a solution comprising a disinfecting agent, the container having an identifier tag identifying the container;
a conduit leading from the container to a manifold;
misting apparatus to generate a mist containing the disinfecting agent and to release the mist via exit conduits, through at least one exit vent; and
a scanner to scan the identifier tag, to identify the container,
wherein the manifold comprises a plenum which receives a volume of air for controlling balance of air pressure as the air is released through the at least one exit vent.

In a preferred embodiment of this aspect of the invention, the tag may be an RFID tag. More preferably, the RFID tag comprises an antenna.

The scanner may comprise an RFID scanner, to scan an RFID tag on the container. However, other types of scanners may be used—for example, the tag may simply be a barcode, and the scanner may be a barcode scanner. In some embodiments, the container identifier may be uploaded to a remote server, for comparison to a container database, to determine whether the container is usable (e.g. not previously used). This helps ensure the integrity and quality of the solution in each container.

In another aspect of the invention, there is provided a disinfection system comprising:
at least one replaceable container, to contain a solution comprising a disinfecting agent, the container having an identifier tag identifying the container;
a conduit leading from the container to at least one exit vent for releasing a mist comprising the disinfection solution;
misting apparatus to generate a mist containing the disinfecting agent and release the mist through the at least one exit vent; and
a capacity sensor to sense the amount of solution in the at least one container.

In a preferred embodiment, there is provided a disinfection system comprising:
at least one replaceable container, to contain a solution comprising a disinfecting agent, the container having an identifier tag identifying the container;
a conduit leading from the container to a manifold;
misting apparatus to generate a mist containing the disinfecting agent and to release the mist via exit conduits, through at least one exit vent; and
   a capacity sensor to sense the amount of solution in the at least one container,
wherein the manifold comprises a plenum which receives a volume of air for controlling balance of air pressure as the air is released through the at least one exit vent.

In a preferred embodiment of this aspect of the invention, the capacity sensor surrounds at least a portion of the container. More preferably, the capacity sensor is fitted on the housing of the disinfection system.

The container may be opaque. Opacity provides advantages in resisting the outgassing of hydrogen peroxide, and in reducing or preventing degradation of the solution due to UV exposure.

Accordingly, the use of a capacity sensor provides the advantage of detecting the amount of solution in the container, when visual inspection is not possible. This is important, because if there is insufficient solution in the container(s), the disinfection system may not adequately disinfect the volume to be disinfected.

The capacity sensor may, for example, comprise internal strip(s) in the housing of the disinfection system, detecting a height of the solution within the container, or may comprise a weight sensor external to the container.

In another aspect of the invention, there is provided a nozzle for connecting a container of disinfecting agent to a disinfection system, the nozzle comprising a clip for securing the container within the nozzle; and an actuator to selectively release the clip so that the container can be removed. The container may have a neck for connection to the nozzle. The neck may have an external screw thread.

The nozzle is preferably rotatable between a storage position and an operative position. Rotation of the nozzle may be manual or electronic.

In another aspect of the invention, there is provided a method of controlling use of a disinfection system, comprising:
scanning a tag associated with a container for a solution containing a disinfecting agent, to obtain an identifier for the container;
enabling or disabling use of the disinfection system based on the identifier.

In this specification, the term ‘comprises’ and its variants are not intended to exclude the presence of other integers, components or steps.

In this specification, reference to any prior art in the specification is not and should not be taken as an acknowledgement or any form of suggestion that this prior art forms part of the common general knowledge in Australia or any other jurisdiction or that this prior art could reasonably expected to be combined by a person skilled in the art.

The present invention will now be more fully described with reference to the accompanying Examples and drawings. It should be understood, however, that the description following is illustrative only and should not be taken in any way as a restriction on the generality of the invention described above.

A detailed description of one or more embodiments of the invention is provided below, along with accompanying figures that illustrate by way of example the principles of the invention. While the invention is described in connection with such embodiments, it should be understood that the invention is not limited to any embodiment. On the contrary, the scope of the invention is limited only by the appended claims and the invention encompasses numerous alternatives, modifications and equivalents.

For the purpose of example, numerous specific details are set forth in the following description in order to provide a thorough understanding of the present invention. The present invention may be practiced according to the claims without some or all of these specific details. For the purposes of clarity, technical material that is known in the technical fields related to the invention has not been described in detail so that the present invention is not unnecessarily obscured.

### BRIEF DESCRIPTION OF THE DRAWINGS/FIGURES

Preferred embodiments of the invention will now be described with reference to the accompanying drawings wherein:

FIG. 1 schematically depicts components of a disinfection regime according to an embodiment of the present invention.

FIG. 2 is a perspective view of a disinfection system according to an embodiment of the invention.

FIG. 3 is a perspective view of internal components of the disinfection system of FIG. 2.

FIG. 4 is a schematic diagram illustrating the relationship and function of the RFID and capacitor sensors in the misting apparatus of the disinfection system.

FIG. 5 is an external view of an exit vent of the disinfection system of FIG. 3.

FIG. 6 is an internal view of the exit vent of FIG. 5.

FIG. 7 is an internal perspective view of the exit vent of FIG. 5.

FIG. 8 is a vertical cross section of the exit vent of FIG. 5.

FIG. 9 is a cross section of the container connected for use in a disinfection system according to FIG. 1, connected using an alternative connection mechanism.

FIG. 10 is a more detailed cross section of the connection of the container as shown in FIG. 9.

FIG. 11 is a plan view of the container when attached to the clip of the disinfection system, taken from the horizontal plane (A-A) as shown in FIG. 10.

FIG. 12 is an external view of a container having an RFID tag attached for label reading.

FIG. 13 is a diagrammatic representation of the possible interactions between various components relative to the PCB.

FIG. 14 is a schematic illustrating the various positions the container may adopt during use of the disinfection system.

### DETAILED DESCRIPTION OF THE EMBODIMENTS

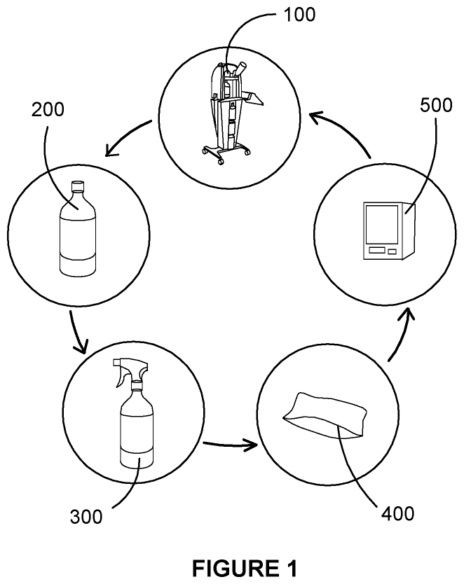

FIG. 1 depicts components for use in a disinfection regime according to an embodiment of the present invention. These include a vapour disinfection system 100, which utilises containers 200 of a solution comprising a disinfecting agent.

The disinfecting agent, in this embodiment, is hydrogen peroxide. Furthermore, in this embodiment, the solution is a 7-8% hydrogen peroxide solution, which may be a substantially pure aqueous solution without silver particles, and where appropriate, without additives such as peracetic acid (although the latter is important when the system is being used in a curative sense rather than a preventative sense). In other embodiments, different disinfecting agents, and/or different solution concentrations may be used.

FIG. 1 further depicts that the solution may be used in spray dispensers 300, or impregnated into disinfectant wipes 400. Finally, an air purifier 500 may also be used for ongoing air purification while a room is in use (e.g. in a dental surgery).

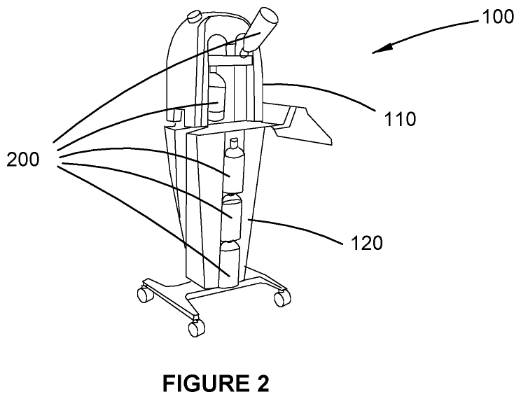

FIG. 2 depicts the disinfection system 100 in more detail. The disinfection system comprises misting apparatus 110 mounted on a trolley 120. Two containers of solution 200 are mountable in the misting apparatus 110, for use in disinfecting the volume of a room.

Compared to conventional misting apparatus, the simultaneous use of two containers 200 of disinfecting solution means that the apparatus will more quickly sanitise the volume of target space.

The trolley 120 includes space to store additional containers 200, as shown in FIG. 2.

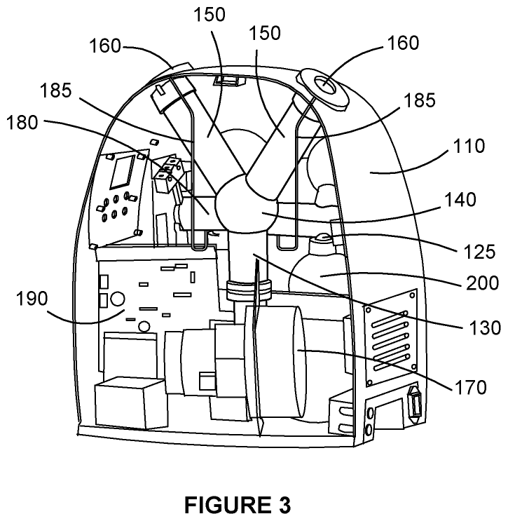

FIG. 3 depicts the internal parts of the misting portion 110 of the system 100 in more detail. The containers 200 are connected, with lids removed, to nozzles 125 which feed into the liquid manifold. In use, the nozzles 125 may be rotated with the liquid manifold to invert the containers 200, to promote flow of hydrogen peroxide solution out of the container and the liquid feed tubes 185, thus providing a controlled supply of hydrogen peroxide that may be dispersed to the surrounding environment via the exit vents 160. More preferably, the supply of hydrogen peroxide is non-pressurised.

The lower conduit 130, plenum 140 and exit conduits 150 together form the air manifold of the disinfection system. The lower conduit 130 may connect the blower 170 to the plenum 140 and the upper conduits may connect the plenum to the exit vents 160.

The lower conduit 130 feeds into a central plenum 140, which has a larger diameter than the lower conduit 130. From there, air pressure created by the blower 170 may be pushed from the plenum 140 into exit conduits 150, and released through exit vents 160. More preferably, the air blower 170 may utilise a motor of 1100 W or 1700 W.

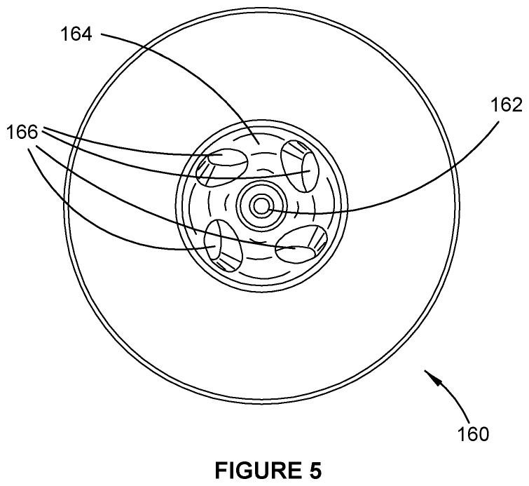

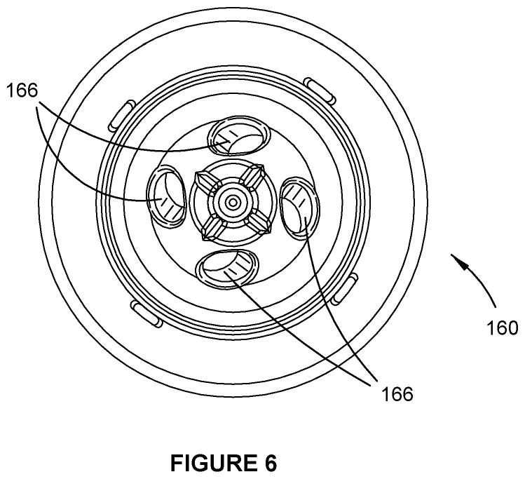

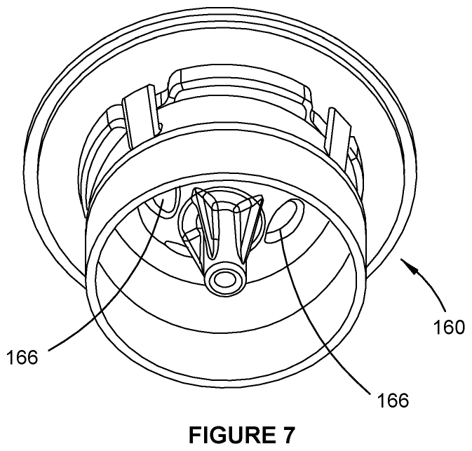

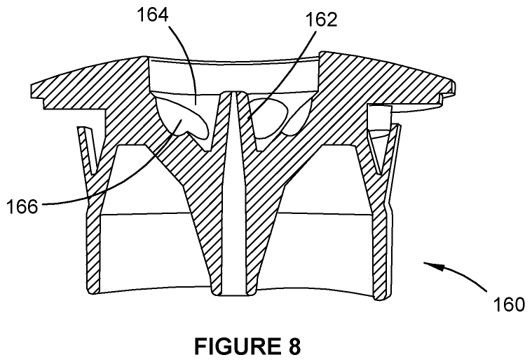

When air pressure is released via the exit conduit 160 a Venturi effect is created, which refers to the suction of a controlled volume of a disinfecting agent from the liquid manifold 180 via the liquid feed tubes 185 to the exit vent 160. The disinfecting agent may then be atomized by the air volume via swirling apertures 166 (FIGS. 5-8) at the exit vent 160, as the air is directed towards the exit nozzle 162 at the appropriate angle.

The plenum 140 provides a volume for air to rectify, before being directed out through exit vents 160. This avoids or mitigates a potential problem with using multiple exit vents 160 —namely, that the air flow may be turbulent and uneven, with greater flow to one side or the other. Thus the plenum 140 assists in controlling the balance and smoothness of air pressure as it is directed towards exit vents 160 via the exit conduits 150. Consequently, this ensures that an equal volume of liquid may be drawn from each container 200. The plenum may further have a role in reducing the occurrence of condensation, which should generally be avoided in the environments where disinfection systems of this type are most commonly deployed.

However, the use of multiple exit vents 160 does allow for greater air flow, thus greatly increasing the rate at which dry mist is created, which allows for rapid disinfection of a target space. Air vents 160 may, in this embodiment, be arranged at 35 degrees from vertical. It has been found that this allows for the benefits of air flow through two exit vents 160, while avoiding or mitigating adverse harmonisation effects, including the occurrence of condensation.

Exit vents 160 are specifically designed to promote the flow of air through vents 160, and to function as a nebulizer so as to atomise the hydrogen peroxide into an efficient particle size for disinfection (between 5 to 15 microns in diameter). Each exit vent includes an external nozzle 162, extending from a recessed bowl 164. Swirling apertures 166 are located around the nozzle 162, enabling the passage of air from the exit conduits 150.

The design of the exit vents 160 utilises the Venturi effect to create hydrogen peroxide mist from the air swirling apertures 166 and the external nozzle 162 for dispersion into the target space to be disinfected. In a preferred embodiment, there may be two exit conduits leading to two exit vents, which may be angled at approximately 35 degrees from the vertical axis of the disinfection system. More preferably, each of the exit vents may comprise a single exit point through which the disinfecting agent may pass.

The misting rate and misting quality may be adjusted by varying factors such as the depth of the bowl 164 and the diameter of the nozzle 162 (FIG. 5). For example, increasing the nozzle 162 diameter and the bowl 164 depth can increase the misting rate but also decrease the misting quality (e.g. may cause the droplet size to be too big). In this embodiment, a preferred nozzle 162 diameter of 1.1 mm is used, and a bowl 164 depth of 12 mm (FIG. 5), to produce appropriately sized droplets at a relatively high misting rate.

In addition, a precisely calibrated choke (not shown) in the liquid feed tube may further control the liquid volume available to the external nozzle 162 to ensure the correct ratio of air-to-liquid to produce optimally sized droplets to form a dry mist.

It is particularly desired for the disinfecting agent to be released by the exit vents 160, having particle sizes of between 5 to 15 microns in diameter, since these sizes influence efficacy of the disinfection system. It is further desired for the disinfecting agent to be released by the exit vents 160 at an appropriate velocity to promote the dry mist to be carried to substantially all parts of the target space to be disinfected. At these particle sizes and velocity, it may be possible for the disinfection system to effectively disinfect a space of metres in longest dimension. In particular, for the disinfection system to effectively disinfect an area of at least 150 cubic metres.

A PCB 190 comprises a microprocessor and a memory, and allows for improved monitoring, control and traceability of the system 100, with the microprocessor providing additional functionality as described in further detail below.

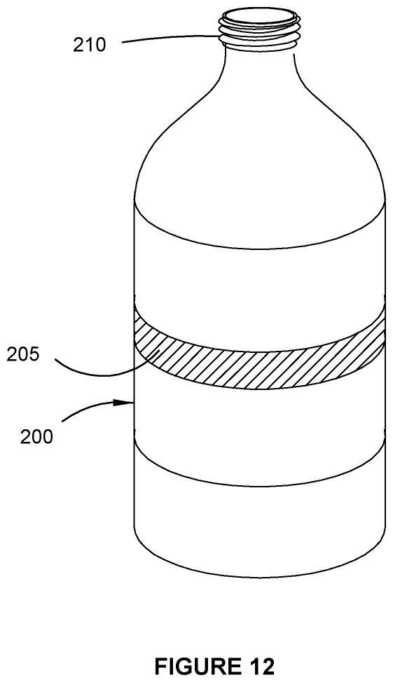

In one embodiment of the present invention, each container 200 includes an RFID tag 205 (FIG. 12). An RFID scanner 118 may be located on misting apparatus 110, either adjacent or within the mounting location of the containers 200, or separately to allow for manual scanning by operators. The RFID scanner 118 may communicate with the microprocessor on PCB 190 via transmitter and receiver antennas, or via cables. The microprocessor may communicate with a remote server, containing a database with information regarding the usage state of each container 200.

When installing a container 200, the operator may be required to scan the RFID tag 205 to identify the container 200 uniquely. The container 200 may then be checked (e.g. in a remote database) to confirm that it has been appropriately filled for use within the disinfection system 100. Each container 200 may only be a single use container—in that way, the supplier of the system can ensure the quality and quantity of the container contents.

If a container 200 is determined to already have been used, the system may reject a further use of this container. This ensures that once a container 200 has had its RFID tag 205 read by the machine, the device will not accept the same container if it is refilled and replaced. If an attempt is made to refill and re-fit a container with a ‘dead’ tag, the device will be inoperable until a container with a ‘live’ tag is put in its place. This will ensure the integrity and quality of the hydrogen peroxide solution.

The RFID tag may have one or more antennae that are programmed to include the details of the manufacturer.

In other embodiments, different types of identifiers (such as bar codes) may be used.

A capacity sensor 116 may also be provided to sense the amount of solution in each container 200. Each container 200 is preferably opaque, to resist the outgassing of hydrogen peroxide, and reduce or prevent degradation of the solution due to UV exposure. Accordingly, the use of a capacity sensor 116 provides the advantage of detecting the amount of solution in the container, when visual inspection is not possible. Again, the capacity sensor 116 may communicate with the microprocessor on PCB 190, to disable the system if there is insufficient solution in the containers 200. This is important, because without sufficient solution, the disinfection system 100 may not adequately disinfect the volume of target space.

The capacity sensor 116 may, for example, comprise internal strip(s) in the housing of the disinfection system, detecting a height of the solution within the container, or may comprise a weight sensor external to the container. More preferably, the capacity sensor 116 comprises multiple sensors that follow a curvature behind the position of a container placed within the disinfection system.

The capacity sensor 116 is designed to accurately measure the volume of disinfecting agent in each container 200. The capacity sensors may allow for regular and iterative measurements to be taken, as the disinfection system is in use. This informs the user on the volume of disinfecting agent remaining in each container 200 at any point in time, whilst also providing in-use, live data, to the PCB 190, in order to perform calculations as discussed below. In carrying out its function, the capacity sensor may further accommodate variables in the container dimensions, materials used in manufacturing the containers and other minor variations that may occur within the container-blowing process, for example in wall thickness.

Since the capacity sensors may measure the amount of disinfecting agent remaining in each container 200, at the end of each operating cycle, the capacity sensors 116 may further assist in limiting the potential for wastage of the disinfecting agent to approximately 3.5% (35 ml) per liter. This may be possible as the disinfection system is capable of computing via the PCB 190 the aggregate amount of remaining disinfecting agent. The user is then subsequently informed via the user interface of the PCB 190, of the relationship between the volume of disinfecting agent remaining (ml) and the largest volume of space (m³) that may be disinfected using that remaining volume (ml).

The user interface of the PCB 190 may display this information on the aggregate amount of remaining disinfectant and on the relationship between the volume of disinfecting agent remaining (ml) and the largest volume of space (m³) that may be disinfected, at the end of the disinfection cycle and also at the beginning of the next operating cycle. This information may also be recorded on the PCB 190, and may be used to direct a user to swap out near-empty containers from the system and to replace these with new containers, thus ensuring that the next operating cycle occurs in a space (m³) that suits the remaining volume of disinfecting agent (ml). Data on wastage and/or swap-out of containers may be recorded to better inform the pattern of usage of the disinfection system. Such information can also be used for the purposes of training users.

The microprocessor on PCB 190 may also electronically control the rotation of containers 200 from a storage position (with nozzles 125 facing down) to an operative position (with nozzles 125 angled upward).

In a preferred embodiment of the invention, efficacy of the disinfection system may be achieved when 7 ml of disinfecting agent is used per m³. Where this optimal amount may be used, the calculations performed by the PCB 190 include the following:
a. Constant 1: 7 ml/m³
b. Constant 2: 0.9 sec/ml
c. Variable 1: m³

Wherein Constant 2 represents the rate at which disinfecting agent is drawn from the disinfection system and is released into the surrounding atmosphere as a dry fog. Taking the two Constants in relation with the Variable, the PCB calculates the time taken to dispense the appropriate volume of disinfecting agent for treating a given space (variable, m³). Thus, the total minutes may be calculated for the operating cycle, based on the following calculations:

((C1×V1)×(C2))÷60=Total minutes for the operating cycle

Where C1=7 ml/m³, V1=60 m³, C2=0.9 sec/ml, and V2=420 ml (C1 (7 ml/m³)×V1 (60 m³)), which gives the following results

((7×60)×(0.9))=378 seconds i.

378 seconds+60=Total minutes for the operating cycle ii.

378+60=6.3 minutes(Total minutes for the operating cycle) iii.

Such information concerning the total minutes for the operating cycle, may be displayed on the user interface of the PCB 190.

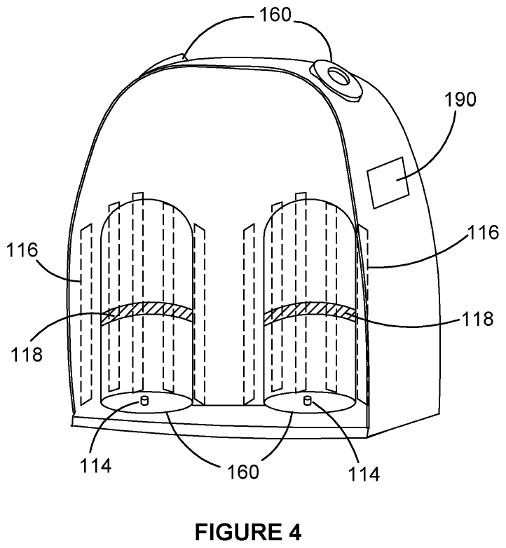

FIG. 4 illustrates some of the key features of the misting apparatus and how these function relative to one another. Specifically, in one embodiment of the present invention, there may be two exit vents 160 on the misting apparatus 110, a floor 112 situated below the lowest point of the container in an upright position, a user interface interactive screen coupled with a PCB 190, and a container stopper 114 fitted on the floor 112 of the misting apparatus. The capacity sensor array 116 and RFID scanner 118 for label reading may be set within the internal curvature of the case of the misting apparatus 110.

The container stopper 114 facilitates each container 200 being positioned at substantially the same distance from the bottom of the capacity sensor array (FIG. 4). More preferably, the container stopper 114 may be positioned in-between the case of the misting apparatus and the container 200. In particular, it may be positioned along the back side of the internal curvature of the misting apparatus case 110. By positioning the container stopper 114 in this way, the capacity sensor 116 may be calibrated, based on a set distance between the container 200 and the RFID scanner 118. Thus, measurements taken by the capacity sensor 116 may be consistent and accurate.

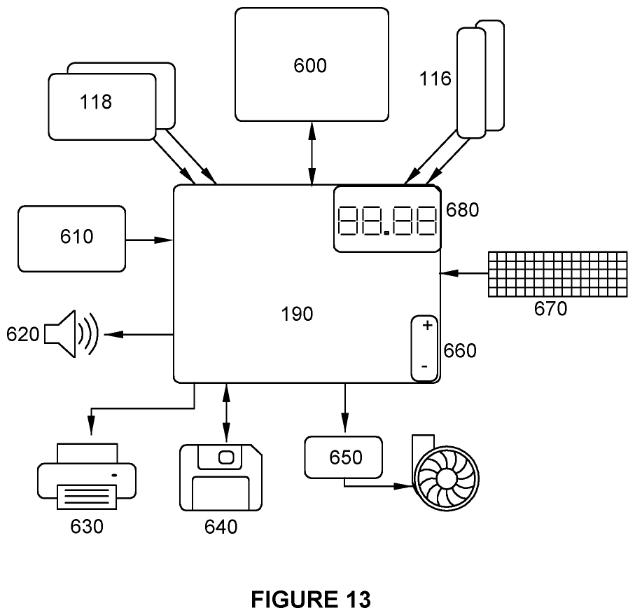

The user interface interactive screen is preferably an LCD screen 600, coupled with a PCB 190 (FIGS. 4 and 13). The presence of the user interface interactive screen is especially desirable in the hospital and medical sectors, since the end-user can observe a measured output and monitor levels of efficacy and efficiency of the disinfection system on a visual basis. Efficiency may be measured in relation to volumes (ml) required of disinfectant per target space (m³). In turn, this information may assist with the management of any potential wastage of the disinfecting agent.

The PCB 190 thus allows for the collection and aggregation of traceability data in relation to operator identity, identification of a target space, date and time of a disinfection treatment, as well as the volume of disinfecting agent that was used in each treatment round. More preferably, data may be collected on maintenance performed on the disinfection system, in addition to any unexpected or unusual use of the system, for example during an outbreak of a specific contamination, bacterial or otherwise. When collecting data on the volume of disinfecting agent used, information on the batch number and date of manufacture of the disinfecting agent may also be recorded.

FIG. 13 demonstrates the various interactions and processes the PCB 190 may carry out in one embodiment of the present invention. The PCB itself may be battery operated 660 and comprise a real-time internal clock 680. The PCB may receive various inputs from the capacity sensor 116, the RFID reader 118, a disinfectant sensor 610, or even a custom keypad 670, which may be attached as a separate peripheral accessory. The PCB may both receive and display information from a LCD screen 600, which may include a graphic user interface, comprising an on-screen keyboard. Data captured by the PCB 190 may be transferred out onto a USB, SD card, or similar storage devices 640, and may also be transferred via Wi-Fi connection (not shown) to a central repository, for example to an infection control department within a hospital. Data may also be sent via Wi-Fi connection, Bluetooth, or similar wireless means, to a printer 630, which may be portable. The printed data may then be retained as a hard copy by the operator and stored in a daily log, or other filing means. Other forms of output via the PCB may include an audio alert 620, and data may also be transferred from a relay 650, which may subsequently be used to control a blower or other similar air conditioning device.

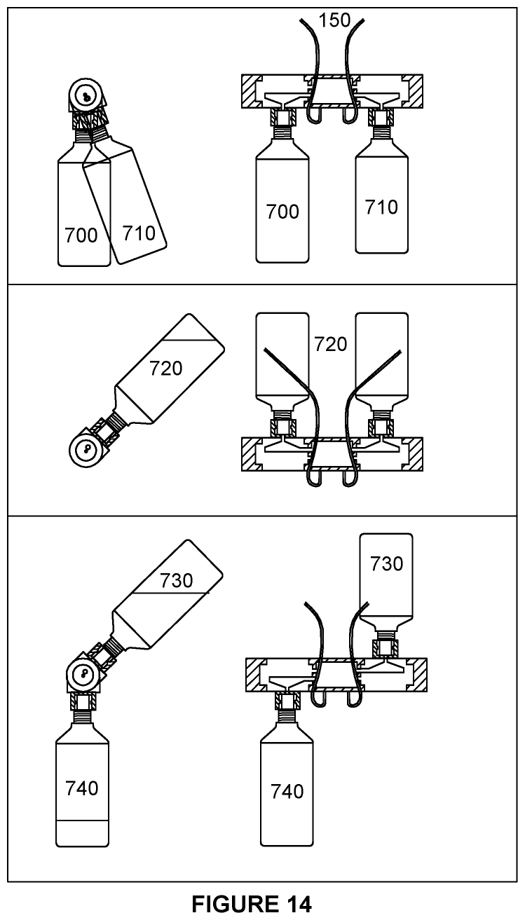

The liquid manifold 180 may have moveable parts to allow the nozzles 125 that receive the containers 200 to be inverted independently of each other (FIG. 3). In a preferred embodiment, the liquid manifold 180 permits the containers 200 to be drained from one to another to reduce or eliminate waste of residual hydrogen peroxide. This may be illustrated by FIG. 14. In the event that the PCB 190 is informed of insufficient aggregate disinfection volumes to disinfect a target room, it may graphically instruct the operator via the LCD screen 600 (FIG. 13) to invert one container, permitting the disinfecting agent to drain from the inverted container 730 to the second receiving container 740. Subsequently, the inverted container emptied of the disinfecting agent may then be returned to the loading position 710, which may be approximately 25° from vertical. From this loading position, the container may be removed and disposed of, such that a new full container may be positioned into the machine for RFID reading and identification 700, such that it may be used by the disinfection system for ongoing disinfection of the target space 720.

Thus, under operative conditions, the container 200 and nozzle 125 may be approximately 35° from horizontal 720 (FIG. 14). Following use of the disinfection system, the containers 200 may be returned to its storage position with nozzles 125 facing down 700 (FIG. 14).

Further in relation to the design of the containers 200, in one embodiment they may comprise a screw seal lid (FIG. 12). The screw thread 210 also allows containers 200 to be engaged with nozzles fitted to lower conduits 130, which may have an internal thread corresponding to thread 210 on the container 200. The screw seal provides an advantage over other types of seal for example, an alloy seal would create a risk of minute particles of alloy coming loose during the piercing of the seal, and would also create a risk for the containers to explode if they were subject to extreme heat in transport, since these types of seal deny any means of escape for the excess gas that might be produced. While the risk of heat-produced gas is extremely low, in some cases pallets of containers may be left outdoors by transport operators or by end users. Accordingly, a vented cap provides a way of reducing the risk of explosion and loss of significant amounts of hydrogen peroxide solution.

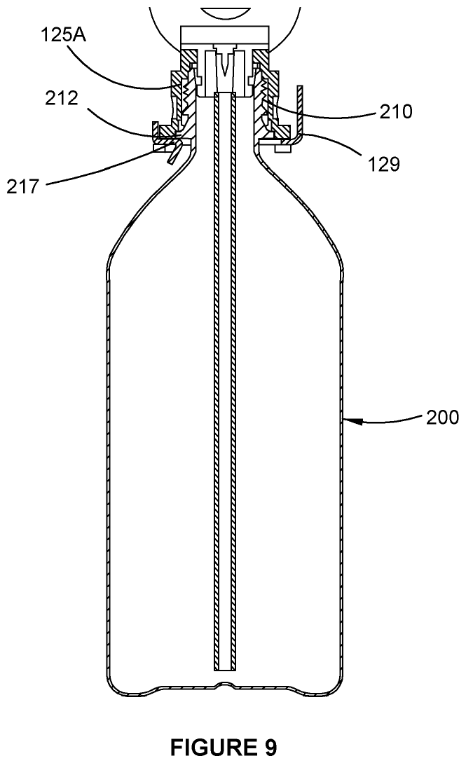

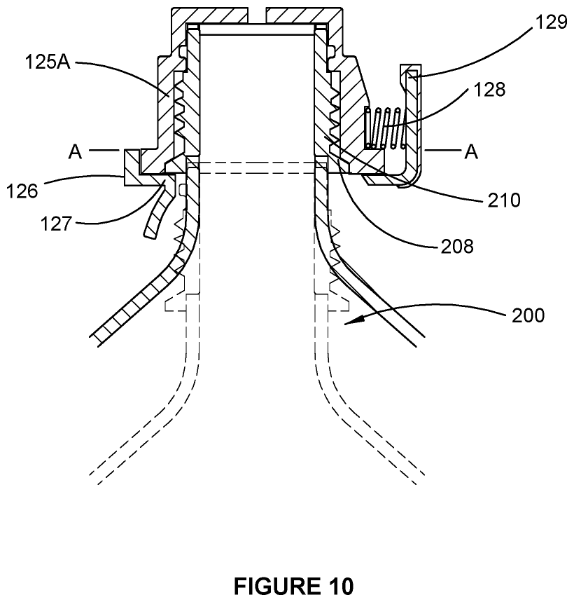

In an alternative embodiment, a clip system may be used to retain the container in nozzle 125, as shown in FIGS. 9 and 10. FIG. 9 depicts an alternative nozzle 125A, which comprises a clip 127, spring 128 and button 129. In use, the clip 127 engages under the bottom collar 212 on the neck of container 200. To install a container 200, the button 129 is depressed, which releases clip 127 by moving it sideways, and allows the neck of container 200 to be inserted into nozzle 125A. When button 129 is released, spring 128 biases button and clip 127 back to a retaining position, where the clip 127 locates under collar 212 on the neck of container 200.

FIGS. 9 and 10 further show that in one embodiment there may be a secondary shoulder on a container 200 to accommodate a security ring (not shown) that may be left on the container, following removal of the container cap. The secondary shoulder centralizes the position, and level of, the security ring, such that it prevents the security ring from potentially interfering with the clip mechanism when attaching the container 200 to the nozzle 125A.

In a preferred embodiment of FIGS. 9 and 10, the height of the tab 126 on the clip 127 may be approximately 8 mm. The preferred width of the neck of the container, when clipped onto the alternative nozzle 125A may be approximately 33.49 mm. The preferred distance between the screw threading on the neck of the container and the button, when released to hold the container in a retaining position, may be approximately 9.29 mm. The preferred angle of slope from the neck of the container to the side of the container may be approximately 45°. Moreover, the preferred angle of the clip tang may be approximately 60° relative to the horizontal plane of the clip.

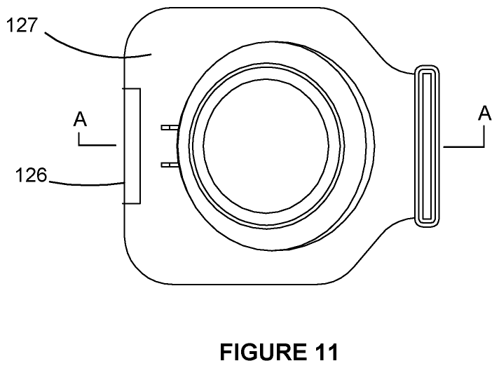

The diameter of clip that allows for insertion of the neck of a container has an aperture that may be sufficiently wide enough to allow the screw threading on the neck of the container to move past the clip without interference, until the secondary shoulder of the container engages with a clip tang. The tab 126 assists with this, as it functions to limit movement of the clip to allow the screw threading on the neck of the container to pass the clip without interference, upon insertion of the container. The clip tang may push back the clip aperture towards maintaining an open position on the clip. In a preferred embodiment of the invention, the clip may have an “O” ring engagement collar, which has an aperture through which the neck of a container may pass through (FIG. 11).

The container 200 may be of variable dimensions, and may be manufactured from different materials, inclusive of PET and HDPE.

The present invention provides numerous advantages over conventional systems. In addition to advantages described previously, it provides an appropriate housing and delivery angle for the Venturi effect to achieve dry fog coverage of rooms requiring sanitizing. It provides consistent tracking and quality assurance of the hydrogen peroxide solution. It reduces the amount of solution wastage, and substantially reduces the time to completely sanitise a 50 cubic metre room to around 6 minutes or less (excluding a 30 minute evaporation period), which is significantly faster than existing systems. Other hydrogen peroxide-based systems heat the hydrogen peroxide, which can create humidification and leave residual moisture. On the other hand, the present invention does not create a heated or steamy vapor because it creates a pulverised “dry fog”, without any condensation, moisture or wet residue being left behind after use of the disinfection system.

The present invention can be deployed across a range of disinfection applications. It is highly relevant to the medical environment, but also has application to other industries, such as: fresh food transportation and storage—extending the shelf life—used in conjunction with air purifiers using photocatalysis and UV light; public transport; childcare centres; general office cleaning environments; animal cages in zoos and veterinary hospitals/clinics; decontamination of abattoirs; food preparation areas; and cruise ships.

Finally, specifically in relation to the air purifier 500, this may utilise needle-point ionization, pulsating negative/positive ion field generator, a corona discharge air fresher and technology comprising UV light and photo-catalyst target, thereby creating an advanced oxidation plasma containing several friendly oxidisers.

The air purifier 500 is best suited for dental surgeries, because it provides a substantial “plug and play” operation, with remote controls to prevent capricious settings changes. The air purifier 500 may be used and kept on during patient treatment systems.

Finally, it is to be understood that various alterations, modifications and/or additions may be made without departing from the spirit of the present invention as outlined herein. These may include the use of applications intended to allow remote starting of the invention and the transmission of relevant, aggregated data from the invention to a mobile device. Furthermore, the system may also use an application for geo-location, to provide operators and supervisors with real-time, accurate information for identifying the whereabouts of the disinfection system.

1. A disinfection system comprising:
a plurality of replaceable containers, to contain a solution comprising a disinfecting agent;
conduits leading from the containers to a manifold;
misting apparatus to generate a mist containing the disinfecting agent and to release the mist via exit conduits, through at least one exit vent,
wherein the manifold comprises a plenum which receives a volume of air for controlling balance of air pressure as the air is released through the at least one exit vent.

2. A disinfection system according to claim 1, wherein the mist is a dry mist.

3. A disinfection system according to claim 2, wherein the dry mist comprises of particles having a diameter size of between approximately 5 and 15 microns.

4. A disinfection system according to claim 1, wherein the manifold comprises the plenum, a lower conduit and exit conduits.

5. A disinfection system according to claim 1, wherein the conduits leading from the containers to the manifold are liquid conduits.

6. A disinfection system according to claim 5, wherein the liquid conduits comprise parts that are independently rotatable relative to one another.

7. A disinfection system according to claim 1, wherein the disinfecting agent is hydrogen peroxide.

8. A disinfection system according to claim 1, wherein the solution is a low dilution solution.

9. A disinfection system according to claim 8, wherein the solution is under 15% hydrogen peroxide.

10. A disinfection system according to claim 9, wherein the solution is in the range of 7% to 8% hydrogen peroxide.

11. A disinfection system according to claim 1, wherein the at least one exit vent comprises a single exit point.

12. A disinfection system according to claim 1, wherein there are at least two exit vents.

13. A disinfection system according to claim 12, wherein the exit conduits are angled at approximately 35 degrees to vertical.

14. A disinfection system comprising:
at least one replaceable container, to contain a solution comprising a disinfecting agent, the container having an identifier tag identifying the container;
a conduit leading from the container to a manifold;
misting apparatus to generate a mist containing the disinfecting agent and to release the mist via exit conduits, through at least one exit vent; and
a scanner to scan the identifier tag, to identify the container,
wherein the manifold comprises a plenum which receives a volume of air for controlling balance of air pressure as the air is released through the at least one exit vent.

15. A disinfection system according to claim 14, wherein the tag is an RFID tag.

16. A disinfection system according to claim 15, wherein the RFID tag comprises an antenna.

17. A disinfection system comprising:
at least one replaceable container, to contain a solution comprising a disinfecting agent, the container having an identifier tag identifying the container;
a conduit leading from the container to a manifold;
misting apparatus to generate a mist containing the disinfecting agent and to release the mist via exit conduits, through at least one exit vent; and
a capacity sensor to sense the amount of solution in the at least one container,
wherein the manifold comprises a plenum which receives a volume of air for controlling balance of air pressure as the air is released through the at least one exit vent.

18. A disinfection system according to claim 17, wherein the capacity sensor surrounds at least a portion of the container.

19. A disinfection system according to claim 18, wherein each container is substantially opaque.

20. A disinfection system according to claim 19, wherein the container has a vented cap.

21. A disinfection system according to claim 20, wherein the container engages with nozzles fitted to a lower conduit of the disinfection system.

22. A method of controlling use of a disinfection system, comprising:
Scanning a tag associated with a container for a solution containing a disinfecting agent, to obtain an identifier for the container; and
enabling or disabling use of the disinfection system based on the identifier.
